(19) United States
(12) Patent Application Publication
(10) Pub. No.: US 2025/0235146 A1
(43) Pub. Date: Jul. 24, 2025

(54) Computer Implemented Method for Determining a Medical Parameter, Training Method and System

(71) Applicant: BIOTRONIK SE & Co. KG, Berlin (DE)

(72) Inventors: Bjoern Henrik DIEM, Berlin (DE); Antje LINNEMANN, Berlin (DE); Anastasia REICH, Berlin (DE)

(73) Assignee: BIOTRONIK SE & Co. KG, Berlin (DE)

(21) Appl. No.: 18/697,791

(22) PCT Filed: Sep. 20, 2022
(86) PCT No.: PCT/EP2022/076013

§ 371 Date: Apr. 2, 2024

(30) Foreign Application Priority Data
Oct. 4, 2021 (EP) 21200656.3

(51) Int. Cl. A61B 5/366; A61B 5/352; A61B 5/355; A61B 5/36

(52) U.S. Cl. CPC A61B 5/366; A61B 5/352; A61B 5/355; A61B 5/36

(57) ABSTRACT

A computer implemented method for determining a QT-interval, a corrected QT-interval or a classification of a QT-interval and/or a classification of a corrected QT-interval, comprising the steps of receiving a first data set comprising pre-acquired cardiac current curve data, in particular one-channel cardiac current curve data, captured by an implantable medical device, applying a machine learning algorithm to the pre-acquired cardiac current curve data, and outputting a second data set representing the QT-interval, the corrected QT-interval or the classification of the QT-interval and/or the corrected QT-interval by the machine learning algorithm. Furthermore, a corresponding system and a method for providing a trained machine learning algorithm is also provided.

### CROSS-REFERENCE TO RELATED APPLICATIONS

This application is the United States National Phase under 35 U.S.C. § 371 of PCT International Patent Application No. PCT/EP2022/076013, filed on Sep. 20, 2022, which claims the benefit of European Patent Application No. 21200656.3, filed on Oct. 4, 2021, the disclosures of which are hereby incorporated by reference herein in their entireties.

### TECHNICAL FIELD

The present invention relates to a computer implemented method for determining a QT-interval, a corrected QT-interval or a classification of a QT-interval and/or a classification of a corrected QT-interval.

### BACKGROUND

Furthermore, the present invention relates to a computer implemented method for providing a trained machine learning algorithm configured to determine a QT-interval, a corrected QT-interval or a classification (C) of a QT-interval and/or a classification of a corrected QT-interval.

In addition, the present invention relates to a system for determining a QT-interval, a corrected QT-interval or a classification of a QT-interval and/or a classification of a corrected QT-interval.

A frequency-corrected QT interval is a medical parameter suitable for early detection of certain drug side effects involving a risk of life-threatening arrhythmias in patients with cardiac implants.

Conventionally, said frequency-corrected QT interval is determined at after care visits of the patient at a health provider, such after care visits typically being scheduled every 1 to 3 months. To this end, a twelve-channel ECG is recorded at the health providers site, based on which the frequency-corrected QT interval may be determined. The recording of a conventional twelve-channel ECG is however associated with a relevant expenditure of time and personnel.

Alternatively, remote transmission of a twelve-channel ECG requires the active cooperation and compliance of the patient, who may be overtaxed. Moreover, one-channel cardiac current curves that may be recorded by means of implantable medical devices are conventionally less suitable for accurately determining said frequency-corrected QT interval when compared to a twelve-channel ECG.

The present disclosure is directed toward overcoming one or more of the above-mentioned problems, though not necessarily limited to embodiments that do.

### SUMMARY

It is therefore an object of the present invention to provide an improved method for early detection of certain drug side effects with risk of life-threatening arrhythmias in patients with cardiac implants capable of providing said early detection more frequently, at reduced cost and increased patient convenience.

At least the object is solved by a computer implemented method for determining a QT-interval, a corrected QT-interval or a classification of a QT-interval and/or a classification of a corrected QT-interval having the features of claim 1.

Furthermore, at least the object is solved by a computer implemented method for providing a trained machine learning algorithm configured to determine a QT-interval, a corrected QT-interval or a classification of a QT-interval and/or a classification of a corrected QT-interval having the features of claim 14.

Moreover, at least the object is solved by a system for determining a QT-interval, a corrected QT-interval or a classification of a QT-interval and/or a classification of a corrected QT-interval having the features of claim 15.

Further developments and advantageous embodiments are defined in the dependent claims.

The present invention provides a computer implemented method for determining a QT-interval, a corrected QT-interval or a classification of a QT-interval and/or a classification of a corrected QT-interval.

The method comprises receiving a first data set comprising pre-acquired cardiac current curve data, in particular one-channel cardiac current curve data, captured by an implantable medical device and applying a machine learning algorithm to the pre-acquired cardiac current curve data.

Furthermore, the method comprises outputting a second data set representing the QT-interval, the corrected QT-interval or the classification of the QT-interval and/or the corrected QT-interval by the machine learning algorithm.

Furthermore, the present invention provides a computer implemented method for providing a trained machine learning algorithm configured to determine a QT-interval, a corrected QT-interval or a classification of a QT-interval and/or a classification of a corrected QT-interval.

The method comprises receiving a first training data set comprising pre-acquired cardiac current curve data, in particular one-channel cardiac current curve data, captured by an implantable medical device and receiving a second training data set representing a QT-interval, a corrected QT-interval or a classification of a QT-interval and/or a classification of a corrected QT-interval.

Moreover, the method comprises training the machine learning algorithm by an optimization algorithm which calculates an extreme value of a loss function for regression of the QT-interval or the corrected QT-interval from the pre-acquired cardiac current curve data or for classification of the QT-interval and/or the corrected QT-interval from the pre-acquired cardiac current curve data.

In addition, the present invention provides a system for determining a QT-interval, a corrected QT-interval or a classification of a QT-interval and/or a classification of a corrected QT-interval.

The system comprises means for receiving a first data set comprising pre-acquired cardiac current curve data, in particular one-channel cardiac current curve data, captured by an implantable medical device and means for applying a machine learning algorithm to the pre-acquired cardiac current curve data.

Furthermore, the system comprises means for outputting a second data set representing the QT-interval, the corrected QT-interval or the classification of the QT-interval and/or the corrected QT-interval by the machine learning algorithm.

An idea of the present invention is to provide automatic remote monitoring of said frequency-corrected QT time for early detection of certain drug side effects with risk of life-threatening arrhythmias in patients with cardiac implants.

The implantable medical device such as an implantable cardiac pacemaker (iLP) or an implantable cardioverter-defibrillator (ICD) regularly records cardiac current curve data and transmits this via a patient device to a central server. There, the frequency-corrected QT-interval (QTc) may be determined from the transmitted data using the machine learning algorithm, and/or the heart rate and the QT-interval may be determined from the transmitted data using the machine learning algorithm and calculated to the frequency-corrected QT-interval (QTc). It is also possible to calculate QTc directly without determining heart rate and QT interval individually.

If this value lies outside the limits set individually for the patient by the physician or if there are changes to the previously transmitted values, the physician is automatically informed via a suitable medium such as e-mail. Thus, an improvement of the quality of therapy with early detection of drug side effects can be achieved.

The machine learning algorithm such as an artificial neural net thus advantageously is able to determine accurate QT- and/or frequency-corrected QT-intervals based on solely a one-channel cardiac current curve.

The cardiac current curve can be, e.g., a subcutaneous ECG, a pseudo-ECG between a shock coil and the implantable medical device or intracardiac current waveforms.

The QT interval is defined from the beginning of the QRS complex to the end of the T wave. The corrected QT interval estimates the QT interval at a standard heart rate of 60 bpm. This allows comparison of QT values over time at different heart rates and improves detection of patients at increased risk of arrhythmias.

The QRS complex is the combination of three of the graphical deflections seen on a typical electrocardiogram (ECG). It is usually the central and most visually obvious part of the tracing. It corresponds to the depolarization of the right and left ventricles of the heart and contraction of the large ventricular muscles.

In adults, the QRS complex normally lasts 80 ms to 100 ms. The Q, R, and S waves occur in rapid succession, do not all appear in all leads, and reflect a single event and thus are usually considered together. A Q wave is any downward deflection usually following the P wave. An R wave follows as an upward deflection, and the S wave is any downward deflection after the R wave. The T wave follows the S wave, and in some cases, an additional U wave follows the T wave.

The RR interval is defined as the time elapsed between two R-waves of successive QRS signal on the electrocardiogram (and its reciprocal, the HR) is a function of intrinsic properties of the sinus node as well as autonomic influences.

Machine learning algorithms are based on using statistical techniques to train a data processing system to perform a specific task without being explicitly programmed to do so. The goal of machine learning is to construct algorithms that can learn from data and make predictions. These algorithms create mathematical models that can be used, for example, to classify data or to solve regression type problems.

According to an aspect of the present invention, if the second data set represents the determined QT-interval, the second data set is used to calculate the corrected QT-interval. The QT-interval determined by the machine learning algorithm can thus advantageously be used to calculate the corrected QT-interval using specific equations such as the Bazett formula: QTC=QT/√RR.

According to a further aspect of the present invention, the machine learning algorithm is a regression-type algorithm, wherein the second data set is given by at least one numeric value, in particular a sequence of numeric values, representing the QT-interval and/or the corrected QT-interval. The machine learning algorithm can thus advantageously either be trained to predict the QT-interval and/or the corrected QT-interval.

According to a further aspect of the present invention, the machine learning algorithm is a classification-type algorithm, wherein the second data set comprises at least one of a first class representing a corrected QT-interval of a normal patient condition and a second class representing a corrected QT-interval of an abnormal patient condition.

This provides the advantage that as opposed to the regression-type algorithm a further step of determining whether or not the predicted QT-interval and/or the corrected QT-interval is within a predetermined range or does not exceed a predetermined threshold value can be omitted since this evaluation is already comprised in the respective classification result.

According to a further aspect of the present invention, the second data set further comprises a third class representing that the corrected QT-interval and/or the classification of the corrected QT-interval is indeterminable from the first data set, in particular from a specific heartbeat of the pre-acquired cardiac current curve data. Should the determined classification represent that the corrected QT-interval is indeterminable from the first data set the result can be discarded, i.e., no notification will be sent to the healthcare provider.

According to a further aspect of the present invention, if at least one value of the second data set representing the corrected QT-interval is outside a predetermined numeric range or is above or below a predetermined threshold value, in particular if the at least one value is outside limits set individually for a patient by a physician and/or if the at least one value differs by a predetermined amount from previously transmitted values, or if the machine learning algorithm classifies a corrected QT-interval of an abnormal patient condition, a notification is sent to a health care provider.

The healthcare provider is thus advantageously informed about a drug side effect potentially involving a risk of life-threatening arrhythmias in a patient with a cardiac implant with reduced delay compared to conventional aftercare visits of the patient.

According to a further aspect of the present invention, the machine learning algorithm is further configured to output a third data set representing a heart rate, wherein the heart rate is determined by detecting an RR interval of a QRS complex of the pre-acquired cardiac current curve data. The machine learning algorithm is thus advantageously configured to predict both the heart rate as well as the QT-interval, and/or the corrected QT-interval.

According to a further aspect of the present invention, the machine learning algorithm determines the QT-interval, the corrected QT-interval or the classification of the QT-interval and/or the corrected QT-interval by detecting a Q-wave and a T-wave and by determining a spacing between the Q-wave and the T-wave of a QRS complex of the pre-acquired cardiac current curve data. The machine learning algorithm is thus advantageously configured to detect the specific patterns of a Q-wave and a T-wave in the cardiac current curve data recorded by the implantable medical device. This machine learning algorithm may also include deep learning approaches that output the correct QT interval but do not directly detect individual Q and T waves.

According to a further aspect of the present invention, a reference value of the QT-interval or the corrected QT-interval obtained by a twelve-channel ECG is compared to the second data set outputted by the machine learning algorithm representing the QT-interval or the corrected QT-interval to calibrate the output of the machine learning algorithm. Said reference value can thus advantageously be used to calibrate the output of the machine learning algorithm to the individual patient.

Based on the reference value of the QT-interval or the corrected QT-interval obtained by the twelve-channel ECG, a most appropriate machine learning algorithm is selected from a library of machine learning algorithms. In this case, and appropriate machine learning algorithm from available multiple machine learning algorithms that matches the individual patient's reference value can be selected.

According to a further aspect of the present invention, the first data set further comprises a thorax impedance and/or a patient activity captured by an implantable medical device. In using said further medical parameters the machine learning algorithm can advantageously generate more accurate results of the output data.

According to a further aspect of the present invention, the cardiac current curve data is acquired by the implantable medical device at predetermined intervals and/or on request, in particular as a wide-field ECG between electrodes and a housing of the implantable medical device, and wherein the cardiac current curve data is transmitted to a central server via a patient communication device or smartphone. It is therefore advantageously not necessary for the patient to perform a multi-channel ECG in a clinical setting. Furthermore, the output data of the algorithm can thus be transmitted to the server for further evaluation according to the predetermined intervals and/or on request thus significantly shortening the time to potentially detect possible drug side effects.

The herein described features of the implantable system for determining a QT-interval, a corrected QT-interval or a classification of a QT-interval and/or a classification of a corrected QT-interval are also disclosed for the computer implemented method for determining a QT-interval, a corrected QT-interval or a classification of a QT-interval and/or a classification of a corrected QT-interval and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a flowchart of a computer implemented method and system for determining a QT-interval QT, a corrected QT-interval QTc or a classification of a QT-interval QT and/or a classification of a corrected QT-interval QTc according to a preferred embodiment of the present invention; and

FIG. 2 shows a flowchart of a computer implemented method for providing a trained machine learning algorithm A configured to determine a QT-interval QT, a corrected QT-interval QTc or a classification of a QT-interval QT and/or a classification of a corrected QT-interval QTc according to the preferred embodiment of the present invention.

### DETAILED DESCRIPTION

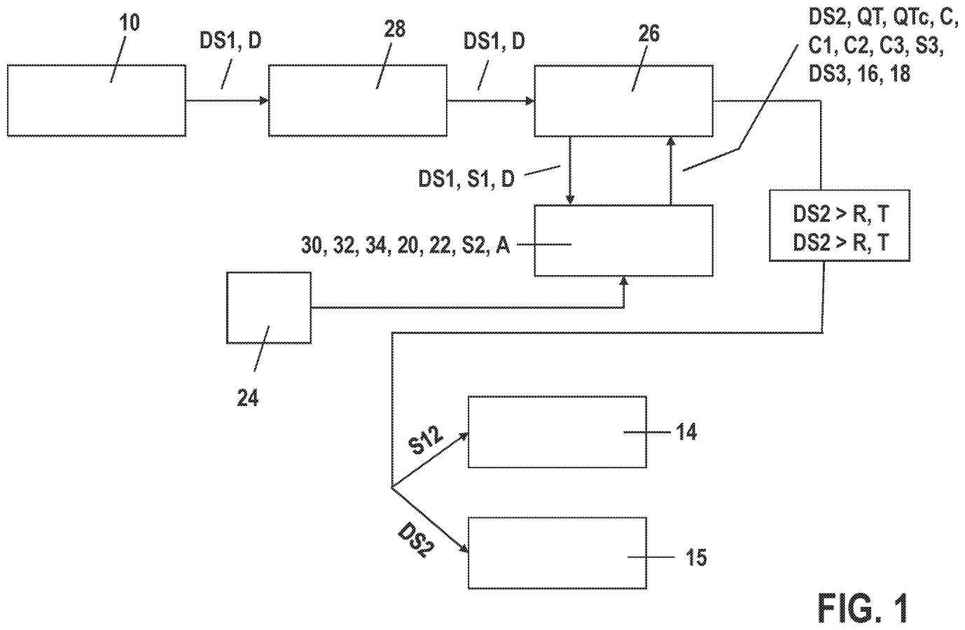

The system for determining a QT-interval QT, a corrected QT-interval QTc or a classification C of a QT-interval QT and/or a classification C of a corrected QT-interval QTc, shown in FIG. 1 comprises means 30 for receiving a first data set DS1 comprising pre-acquired cardiac current curve data D, in particular one-channel cardiac current curve data D, captured by an implantable medical device 10.

Furthermore, the system comprises means 32 for applying a machine learning algorithm A to the pre-acquired cardiac current curve data D and means 34 for outputting a second data set DS2 representing the QT-interval QT, the corrected QT-interval QTc or the classification C of the QT-interval QT and/or the corrected QT-interval QTc by the machine learning algorithm A.

If the second data set DS2 represents the determined QT-interval QT, the second data set DS2 is used to calculate the corrected QT-interval QTc. The machine learning algorithm A is preferably a regression-type algorithm, wherein the second data set DS2 is given by at least one numeric value, in particular a sequence of numeric values, representing the QT-interval QT and/or the corrected QT-interval QTc.

Alternatively, the machine learning algorithm A can be embodied as a classification-type algorithm, wherein the second data set DS2 comprises at least one of a first class C1 representing a corrected QT-interval QTc of a normal patient condition and a second class C2 representing a corrected QT-interval QTc of an abnormal patient condition.

The second data set DS2 may further comprise a third class C3 representing that the corrected QT-interval QTc and/or the classification of the corrected QT-interval is indeterminable from the first data set DS1, in particular from a specific heartbeat of the pre-acquired cardiac current curve data D.

If at least one value of the second data set DS2 representing the corrected QT-interval QTc is outside a predetermined numeric range R or is above or below a predetermined threshold value V, in particular if the at least one value is outside limits set individually for a patient by a physician and/or if the at least one value differs by a predetermined amount from previously transmitted values, or if the machine learning algorithm A classifies a corrected QT-interval QTc of an abnormal patient condition, a notification 12 is sent to a communication device 14 of a health care provider in order to alarm the health care provider of the at least one abnormal value of the second data set DS2.

Said notification 12 is preferably sent by e-mail. Alternatively, the notification 12 may be sent by text message (SMS) or by means of an in-app notification. Furthermore, the healthcare provider may access the at least one value of the second data set DS2 representing the corrected QT-interval QTc via a front-end application 15 on a suitable communication device such as a smart phone and/or a personal computer.

The machine learning algorithm A is further configured to output a third data set DS3 representing a heart rate, wherein the heart rate is determined by detecting an RR interval 16 of a QRS complex 18 of the pre-acquired cardiac current curve data D.

If the machine learning algorithm A outputs the second data set DS2 representing the QT-interval QT, the corrected QT-interval QTc is calculated based on the QT-interval QT and the heart rate. The machine learning algorithm A determines the QT-interval QT, the corrected QT-interval QTc or the classification C of the QT-interval QT and/or the corrected QT-interval QTc by detecting a Q-wave 20 and a T-wave 22 and by determining a spacing between the Q-wave 20 and the T-wave 22 of the QRS complex 18 of the pre-acquired cardiac current curve data D.

A reference value 24 of the QT-interval QT or the corrected QT-interval QTc obtained by a twelve-channel ECG is compared to the second data set DS2 outputted by the machine learning algorithm A representing the QT-interval QT or the corrected QT-interval QTc to calibrate the output of the machine learning algorithm A.

Based on the reference value 24 of the QT-interval QT or the corrected QT-interval QTc obtained by the twelve-channel ECG, a most appropriate machine learning algorithm A is selected from a library of machine learning algorithms A. The first data set DS1 further comprises a thorax impedance and/or a patient activity captured by an implantable medical device 10.

The cardiac current curve data D is acquired by the implantable medical device 10 at predetermined intervals and/or on request, in particular as a wide-field ECG between electrodes and a housing of the implantable medical device 10, and wherein the cardiac current curve data D is transmitted to a central server 26 via a patient communication device 28 or smartphone.

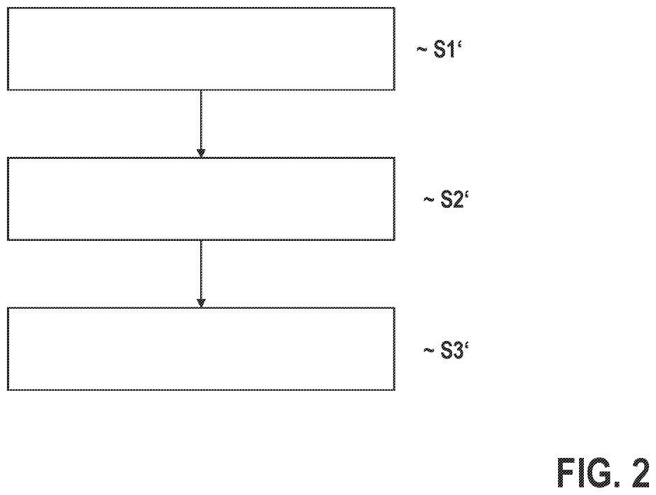

FIG. 2 shows a flowchart of a computer implemented method for providing a trained machine learning algorithm A configured to determine a QT-interval QT, a corrected QT-interval QTc or a classification C of a QT-interval QT and/or a classification C of a corrected QT-interval QTc according to the preferred embodiment of the present invention.

The method comprises receiving S1′ a first training data set comprising pre-acquired cardiac current curve data D, in particular one-channel cardiac current curve data D, captured by an implantable medical device 10.

Furthermore, the method comprises receiving S2′ a second training data set representing a QT-interval QT, a corrected QT-interval QTc or a classification C of a QT-interval QT and/or a classification C of a corrected QT-interval QTc.

In addition, the method comprises training S3′ the machine learning algorithm A by an optimization algorithm which calculates an extreme value of a loss function for regression of the QT-interval QT or the corrected QT-interval QTc from the pre-acquired cardiac current curve data D or for classification C of the QT-interval QT and/or the corrected QT-interval QTc from the pre-acquired cardiac current curve data D.

The machine learning algorithm A configured to determine a QT-interval QT, a corrected QT-interval QTc or a classification C of a QT-interval QT and/or a classification C of a corrected QT-interval QTc is trained using corresponding pairs of the first training data set and the second training data set.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

10 implantable medical device
12 notification
14 communication device
15 front-end application
16 RR interval
18 QRS complex
20 Q-wave
22 T-wave
24 reference value
26 central server
28 patient communication device
30 means
32 means
34 means
A machine learning algorithm
C classification
C1 first class
C2 second class
C3 third class
D cardiac current curve data
DS1 first data set
DS2 second data set
QT QT-interval
QTc corrected QT-interval
R predetermined numeric range
S1-S3 method steps
S1′-S3′ method steps
V threshold value

1. Computer implemented method for determining a QT-interval, a corrected QT-interval or a classification of a QT-interval and/or a classification of a corrected QT-interval, comprising the steps of:
receiving a first data set comprising pre-acquired cardiac current curve data, in particular one-channel cardiac current curve data, captured by an implantable medical device;
applying a machine learning algorithm to the pre-acquired cardiac current curve data; and
outputting a second data set representing the QT-interval, the corrected QT-interval or the classification of the QT-interval and/or the corrected QT-interval by the machine learning algorithm.

2. Computer implemented method of claim 1, wherein if the second data set represents the determined QT-interval, the second data set is used to calculate the corrected QT-interval.

3. Computer implemented method of claim 1, wherein the machine learning algorithm is a regression-type algorithm, wherein the second data set is given by at least one numeric value, in particular a sequence of numeric values, representing the QT-interval and/or the corrected QT-interval.

4. Computer implemented method of claim 1, wherein the machine learning algorithm is a classification-type algorithm, wherein the second data set comprises at least one of a first class representing a corrected QT-interval of a normal patient condition and a second class representing a corrected QT-interval of an abnormal patient condition.

5. Computer implemented method of claim 4, wherein the second data set further comprises a third class representing that the corrected QT-interval and/or the classification of the corrected QT-interval is indeterminable from the first data set, in particular from a specific heartbeat of the pre-acquired cardiac current curve data.

6. Computer implemented method of claim 1, wherein if at least one value of the second data set representing the corrected QT-interval is outside a predetermined numeric range or is above or below a predetermined threshold value, in particular if the at least one value is outside limits set individually for a patient by a physician and/or if the at least one value differs by a predetermined amount from previously transmitted values, or if the machine learning algorithm classifies a corrected QT-interval of an abnormal patient condition, a notification is sent to a communication device of a health care provider.

7. Computer implemented method of claim 1, wherein the machine learning algorithm is further configured to output a third data set representing a heart rate, wherein the heart rate is determined by detecting an RR interval of a QRS complex of the pre-acquired cardiac current curve data.

8. Computer implemented method of claim 7, wherein if the machine learning algorithm outputs the second data set representing the QT-interval, the corrected QT-interval is calculated based on the QT-interval and the heart rate.

9. Computer implemented method of claim 7, wherein the machine learning algorithm determines the QT-interval, the corrected QT-interval or the classification of the QT-interval and/or the corrected QT-interval by detecting a Q-wave and a T-wave and by determining a spacing between the Q-wave and the T-wave of the QRS complex of the pre-acquired cardiac current curve data.

10. Computer implemented method of claim 1, wherein a reference value of the QT-interval or the corrected QT-interval obtained by a twelve-channel ECG is compared to the second data set outputted by the machine learning algorithm representing the QT-interval or the corrected QT-interval to calibrate the output of the machine learning algorithm.

11. Computer implemented method of claim 10, wherein based on the reference value of the QT-interval or the corrected QT-interval obtained by the twelve-channel ECG, a most appropriate machine learning algorithm is selected from a library of machine learning algorithms.

12. Computer implemented method of claim 1, wherein the first data set further comprises a thorax impedance and/or a patient activity captured by an implantable medical device.

13. Computer implemented method of claim 1, wherein the cardiac current curve data is acquired by the implantable medical device at predetermined intervals and/or on request, in particular as a wide-field ECG between electrodes and a housing of the implantable medical device, and wherein the cardiac current curve data is transmitted to a central server via a patient communication device or smartphone.

14. Computer implemented method for providing a trained machine learning algorithm configured to determine a QT-interval, a corrected QT-interval or a classification of a QT-interval and/or a classification of a corrected QT-interval, comprising the steps of:
receiving a first training data set comprising pre-acquired cardiac current curve data, in particular one-channel cardiac current curve data, captured by an implantable medical device;
receiving a second training data set representing a QT-interval, a corrected QT-interval or a classification of a QT-interval and/or a classification of a corrected QT-interval; and
training the machine learning algorithm by an optimization algorithm which calculates an extreme value of a loss function for regression of the QT-interval or the corrected QT-interval from the pre-acquired cardiac current curve data or for classification of the QT-interval and/or the corrected QT-interval from the pre-acquired cardiac current curve data.

15. System for determining a QT-interval, a corrected QT-interval or a classification of a QT-interval and/or a classification of a corrected QT-interval, comprising:
means for receiving a first data set comprising pre-acquired cardiac current curve data, in particular one-channel cardiac current curve data, captured by an implantable medical device;
means for applying a machine learning algorithm to the pre-acquired cardiac current curve data; and
means for outputting a second data set representing the QT-interval, the corrected QT-interval or the classification of the QT-interval and/or the corrected QT-interval by the machine learning algorithm.
